(19) United States
(12) Patent Application Publication
(10) Pub. No.: US 2013/0274846 A1
(43) Pub. Date: Oct. 17, 2013

(54) METHODS, DEVICES AND SYSTEMS FOR TREATING PAIN

(71) Applicant: NeuroAccess Technologies, Boston, MA (US)

(72) Inventors: Shivanand Lad, Durham, NC (US); Scott Kokones, Brookline, MA (US); Gregory Schulte, Minneapolis, MN (US); Andrew Firlik, Darien, CT (US); Vincent Owens, Hingham, MA (US)

(21) Appl. No.: 13/834,997

(22) Filed: Mar. 15, 2013

Related U.S. Application Data
(60) Provisional application No. 61/622,337, filed on Apr. 10, 2012.
(60) Provisional application No. 61/712,517, filed on Oct. 11, 2012.
(60) Provisional application No. 61/729,452, filed on Nov. 23, 2012.

(51) Int. Cl. A61N 1/05

(52) U.S. Cl. CPC A61N 1/0551
USPC 607/117

(57) ABSTRACT

Methods and devices for stimulating the spinal cord are provided. The method involves positioning an electrical lead in the intrathecal space adjacent to the desired level of the spinal cord. Electrical leads are provides that help stabilize the lead in the intrathecal space and provide for focal stimulation of the spinal cord.

### CROSS REFERENCE TO RELATED APPLICATIONS

The present application claims priority to U.S. Provisional Application No. 61/622,337 filed on Apr. 10, 2012 entitled: “Systems and Methods for Stimulating the Spinal Cord;” U.S. Provisional Application No. 61,712,517 filed on Oct. 11, 2012 entitled: “Lead Assemblies, Implantation Tools, Accessory Tools, and Systems and Methods of Using Same;” and U.S. Provisional Application No. 61/729,452 filed on Nov. 23, 2012 entitled: “Positioning Elements and Tools for Adding Positioning Elements to Leads and Catheters,” all of which are incorporated by reference herein in their entirety.

### TECHNICAL FIELD

The present invention relates to systems, devices and methods for treating different types of pain and other medical conditions by intrathecal stimulation of the spinal cord.

### BACKGROUND

Spinal cord stimulation (SCS) has been used to treat chronic painful conditions for approximately thirty years. Commonly, SCS is used to alleviate pain after failed back surgery, pain due to neuropathies, or pain due to inadequate blood flow. Physicians routinely use two different SCS systems: those involving percutaneously placed electrical leads and those requiring laminectomies to allow placement of an electrical lead. The first system involves percutaneous insertion of an electrical lead into the epidural space and either transcutaneous connection to an external generator, allowing a trial period of stimulation, or subcutaneous connection to an implanted radio frequency-controlled receiver or an implantable pulse generator. The second system requires implantation of a paddle-type electrical lead into the epidural space after a laminectomy. Similar to percutaneously placed lead, the electrical paddle-type lead may be connected to an external generator, allowing a trial period of stimulation, or may be connected subcutaneously to a radio frequency receiver or an implantable pulse generator. The radio frequency receiver is activated by an external battery-powered transmitter, which operates through an antenna placed over the receiver. The implantable pulse generator contains a battery that supplies power to the electrodes of the lead. (See Tracy Cameron, “Safety and efficacy of spinal cord stimulation for the treatment of chronic pain: a 20 year literature review,” J. Neurosurg (Spine 3), 100:254-267 (2004)).

Several problems can arise from implantation of an electrical lead in the epidural space. For example, because the electrical lead is placed outside of the dura, there is significant distance between the electrical lead and the spinal cord. As such, the electrical current must penetrate the dura and other layers of the spinal cord thereby requiring amplitudes that are high in order to effectively stimulate the relevant spinal cord fibers. Cerebrospinal fluid is the primary buffer in epidural stimulation, resulting in shunting of approximately 90% of the electrical current from the intended target. This has been a primary deficiency of spinal cord stimulation and resulting in the need for higher amplitudes and spread of current to dorsal root ganglia and nerve roots. In particular, electrical current can spread to unintended regions of the spinal cord and spinal nerves causing paresthesias in parts of the body other than the desired dermatome.

Intradural or intrathecal stimulation provides for more direct and targeted stimulation of the relevant fibers of the spinal cord thereby reducing the side effects associated with epidural stimulation. In particular, lower amplitude and more focal stimulation allows for stimulation of the relevant fibers without stimulation of fibers that can result in painful or undesirable consequences. In addition to less side effects, placing the electrical lead intradurally allows for easier lead placement due to the lack of scar tissue in the intrathecal space. It can be difficult to place percutaneous leads in the epidural space for patients who have had previous back surgeries, for example, because of the scar tissue that forms in the epidural space. Furthermore, up to fifty percent of patients who have initially benefited from epidural stimulation become refractory after three to five years. Although not wishing to be bound by theory, it is believed that this is the result of thickened arachnoid/dural scar formation that results in an increasing voltage requirement and eventually unwanted side effects of stimulation.

Although intrathecal stimulation of the spinal cord reduces stimulation levels compared to epidural stimulation and provides for more focal stimulation of the spinal cord, there are also issues associated with this route of stimulation. For example, intrathecal lead placement can cause cerebrospinal fluid leakage. Further, percutaneous leads that can be used for intrathecal stimulation are prone to movement and migration. Therefore, a more effective method of intrathecally stimulating the spinal cord is needed.

### SUMMARY

The present invention provides systems, methods and devices for intrathecally stimulating the spinal cord to treat certain medical conditions or improve certain functions in a patient suffering from such medical conditions or having a deficit of such functions. In an embodiment, the present invention provides a method of treating pain in a patient suffering therefrom. The method comprises identifying a patient suffering from the pain. The method further comprises inserting an electrical lead into the patient's body, positioning the electrical lead in the patient's intrathecal space at a level of the spinal cord, delivering an electrical signal to the patient's spinal cord at the spinal cord level, and treating the pain in the patient. The pain is axial back pain, groin pain or cancer pain.

In another embodiment, the present invention provides a method of improving sensory or motor function in a patient suffering from a medical condition comprising identifying a patient suffering from the medical condition. The method then comprises inserting an electrical lead into the patient's body, positioning the electrical lead in the patient's intrathecal space at a level of the spinal cord, delivering an electrical signal to the patient's spinal cord at the spinal cord level to modulate a spinal cord pathway and improving the sensory or motor function in the patient. The medical condition can be neuropathic pain, nociceptive pain, stroke, traumatic brain injury, or ataxia.

In another embodiment, the present invention provides several different types of electrical leads that are suitable for intrathecal stimulation. In an embodiment, the present invention provides an electrical lead comprising a lead body having a proximal portion comprising a plurality of electrical contacts disposed thereon and a distal portion comprising a plurality of electrodes disposed thereon. The electrical lead further includes a slotted plug preferably located between the proximal portion and the distal portion. In certain embodiments, the slotted plug has a maximum outer diameter greater than the maximum outer diameter of the lead body.

In another embodiment, the present invention provides an electrical lead comprising a lead body having a distal portion. The lead further includes a cover disposed less than 360 degrees about the distal portion of the lead body. The lead also includes a plurality of electrodes circumferentially disposed on the distal portion of the lead body. Each of the plurality of electrodes has an exposed region and a shielded region facing the cover. In certain embodiments, the electrodes comprise notches, each defining a bore extending in an axis different than the longitudinal axis of the lead body.

In another embodiment, the present invention provides an electrical lead comprising a lead body having a distal portion. The lead includes an overtube disposed 360 degrees about the distal portion of the lead body and defining a plurality of apertures. The lead also comprises a plurality of electrodes disposed on the distal portion of the lead body. Each of the plurality of electrodes has a shielded region and an exposed region, the exposed region aligned with a respective one of the plurality of apertures of the overtube. The plurality of electrodes can be circumferentially disposed about the distal portion of the lead body and can have a diameter smaller than the diameter of the lead body.

The exposed region of each of the plurality of electrodes can be recessed in the overtube. The electrodes can comprise notches, each notch defining a bore extending in an axis different than the longitudinal axis of the lead body. In certain embodiments, each of the plurality of electrodes has a raised outer surface flush with the outer surface of the overtube. In other embodiments, each of the plurality of electrodes has a raised outer surface such that each of the electrodes has a maximum outer diameter greater than the maximum outer diameter of the overtube. The raised surface of each of the electrodes protrudes out of the respective one of the plurality of apertures of the overtube.

In another embodiment, the present invention provides an electrical lead comprising a lead body having a distal portion comprising a plurality of electrodes and a plurality of arms extending from the lead body having a compressed configuration and a relaxed configuration. The plurality of arms is oriented radially outwards in a relaxed configuration. The distal portion of the lead body has a distal end and the plurality of arms can be disposed on the distal end of the distal portion.

In another embodiment, the present invention provides an electrical lead comprising a lead body having a distal portion comprising a plurality of electrodes. The electrical lead further comprises at least three arms extending from the lead body having a compressed configuration and a relaxed configuration. At least two of the at least three arms are separated by greater than 120 degrees in a relaxed configuration. The distal portion of the lead body has a distal end and the at least three arms can be disposed on the distal end of the distal portion.

In another embodiment, the present invention provides an electrical lead comprising a lead body having a distal portion comprising a plurality of electrodes. The electrical lead further includes

a fin coupled directly or indirectly to the lead body and extending laterally from the lead body. The electrical lead can further include a cover disposed less than 360 degrees about the distal portion of the lead body. The fin can be coupled directly to the cover.

### BRIEF DESCRIPTION OF THE DRAWINGS

FIG. 1 is a side view of an electrical lead according to an embodiment of the present invention.

FIG. 2 is a side view of a plug of the electrical lead depicted in FIG. 1.

FIG. 3 is a perspective view of an electrical lead according to an embodiment of the present invention.

FIG. 4 is a perspective view of an electrical lead according to an embodiment of the present invention.

FIG. 5 is a cross-sectional view of the distal portion of the electrical lead of FIG. 4.

FIG. 6 is a perspective view of an electrical lead according to an embodiment of the present invention.

FIG. 7 is an exploded view of the electrical lead of FIG. 6 with a cover according to an embodiment of the present invention.

FIG. 8 is a cross-sectional view of the distal portion of the electrical lead of FIG. 3.

FIG. 9 is a cross-sectional view of the distal portion of an electrical lead according to an embodiment of the present invention.

FIG. 10 is an end view of the distal portion of the electrical lead of FIG. 11.

FIG. 11 is a perspective view of an electrical lead according to an embodiment of the present invention.

FIG. 12 is a perspective view of an electrode according to an embodiment of the present invention.

FIG. 13 is a perspective view of an electrical lead comprising the electrode of FIG. 12 according to an embodiment of the present invention.

FIG. 14 is a perspective view of an electrical lead according to an embodiment of the present invention.

FIG. 15 is a side view of an electrical lead according to an embodiment of the present invention.

FIG. 16 is a side view of an electrical lead according to an embodiment of the present invention in a compressed configuration.

FIG. 17 is a perspective view of the electrical lead of FIG. 16 in a relaxed or expanded configuration.

FIG. 18 is a perspective view of an electrical lead according to an embodiment of the present invention.

FIG. 19 is a perspective back view of an electrical lead according to an embodiment of the present invention.

FIG. 20 is a perspective front view of an electrical lead according to an embodiment of the present invention.

FIG. 21 is a perspective back view of an electrical lead according to an embodiment of the present invention.

### DETAILED DESCRIPTION

The disclosure herein may refer to electrical or neural “stimulation” or “modulation.” Such terms include inhibition or activation of electrical activity in and/or around the therapy site. The disclosure herein also refers to “treating” certain medical conditions. This does not necessarily mean curing the medical condition but includes improving or minimizing the patient's symptoms. The disclosure herein also refers to the term “substantially” with respect to certain geometric shapes, configurations and orientations. By “substantially” is meant that the shape, configuration or orientation of the element need not have the mathematically exact described shape, configuration or orientation but can have a shape, configuration or orientation that is recognizable by one skilled in the art as generally or approximately having the described shape, configuration, or orientation. Also, the disclosure herein refers to an “operative configuration.” This is the configuration of the system when the medical device has been inserted into the patient and is implanted in the target site. Further, as used herein with respect to a described element, the terms “a,” “an,” and “the” include at least one or more of the described element unless otherwise indicated. Further, the term “or” refers to “and/or” unless otherwise indicated. In addition, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an is referred to as being, for example, “directly on,” “directly attached” to, “directly connected” to,“directly coupled” with or “directly contacting” another element, there are no intervening elements present. It will also be appreciated by those of skill in the art that references to an element that is disposed “adjacent” another element may have portions that overlap or underlie the adjacent element.

The present invention is directed to intrathecal stimulation of the spinal cord to treat pain and other medical conditions in a patient suffering therefrom. In preferred embodiments, the patient is a mammal, such as a human being. In certain embodiments, the present invention provides methods of treating chronic axial back pain. In other embodiments, the present invention provides methods for treating groin pain. In still other embodiments, the present invention provides methods for treating cancer pain. In other embodiments, the present invention provides methods of improving functionalities in patients suffering from other conditions such as neuropathic pain, nociceptive pain, stroke, traumatic brain injury (TBI) or ataxia.

Regarding axial pain, axial back pain is primarily centered in the low back area. In certain embodiments, the present invention relates to treating back pain without a radicular component (i.e. treating back pain in a patient who does not suffer from radicular pain). Radicular pain is pain that radiates in the lower extremity directly along the course of a spinal nerve root. In other embodiments, the patient suffers from radicular pain but back pain is the pain for which the patient is primarily seeking treatment.

The present invention also provides methods for stimulating certain spinal cord pathways to improve motor or sensory functions in patients suffering from certain medical conditions. Table I below identifies the relevant pathways, the functionalities that these pathways regulate, and the clinical indications for stimulating these pathways.

TABLE I

| Pathway | Function | Clinical Indications |
|---|---|---|
| Lateral | Pain and temperature | Neuropathic and |
| spinothalamic | | Nociceptive Pain |
| Anterior | Pain and temperature | Neuropathic and |
| spinothalamic | | Nociceptive Pain |
| Fasciculus | Touch, pressure and conscious | Neuropathic Pain |
| gracile | muscle joint sense | |
| Fasciculus | Touch, pressure and conscious | Neuropathic Pain |
| cuneate | muscle joint sense | |
| Dorsal | Proprioception | Stroke; TBI; Ataxi |
| spinocerebellar | | |
| Ventral | Provide input to the cerebellum | Stroke; TBI; Ataxi |
| spinocerebellar | | |
| Lateral | Voluntary skilled movement of | Stroke; TBI |
| corticospinal | trunk and hind limbs | |
| Ventral | Voluntary skilled movement of | Stroke; TBI |
| corticospinal | forelimb | |
| Tectospinal | Reflex postural movement in | Stroke; TBI |
| | response to visual stimuli | |
| Rubrospinal | Facilitate activity of flexor muscles | Stroke; TBI |
| | and inhibits extensors | |
| Vestibulospinal | Facilitates activity of extensor | Stroke; TBI |
| tract | muscles and inhibition of flexor | |
| | muscles under the influence of the | |
| | ear and cerebellum in maintenance | |
| | of balance | |

In particular, in an embodiment, the present invention provides a method of treating certain medical conditions or improving certain functions comprising identifying a patient suffering from the respective condition or having a deficit of the respective function. After the suitable patient has been identified, the method comprises positioning an electrical lead in the patient's intrathecal space at the appropriate spinal level, such as the thoracic spinal level for axial back pain, and delivering an electrical signal to the patient's spinal cord at the appropriate spinal level to treat the medical condition or improve the function.

Regarding the step of identifying a patient suffering from axial back pain, there are several different ways in which a suitable patient can be identified. For example, there are well-known patient questionaires and clinician guidelines that can be used to determine if a patient suffers from axial back pain (See e.g. McGill Pain Questionaire; painDetect® pain questionaire by Pfizer Pharma GmbH; Initial Pain Assessment Tool (Pasero C, McCaffery M, Pain: Assessment and pharmacologic management (2011), Mosby, Inc.); Initial Pain Assessment (adapted from Management of Cancer Pain, Clinical Guidelines No. 9 AHCPR Publication No. 94-0592: March 1994. Agency for Healthcare Research and Quality, Rockville, Md.); Brief Pain Inventory (Charles S. Cleeland), “Guidelines for the Evaluation and Management of Low Back Pain,” R. Chou and L. Hoyt Huffman (American Pain Society, Glenview, Ill.)), all of which are incorporated by reference in their entirety. Generally, a patient suffering from axial back pain has low back pain that gets worse when performing certain activities and when assuming certain positions such as sitting down for long periods of time. In fact, such a patient may feel better by walking or even running than sitting or standing. The low back pain is also generally relieved by rest or changing positions frequently. The patient may also suffer from low-levels of constant lower back pain punctuated by episodes of severe pain/muscle spasms lasting a few days to a few months. The chronic pain can range from nagging to severe.

Once a patient suffering from the medical condition or deficit function has been identified, a method of the present invention includes performing SCS on the patient by positioning an electrical lead in a patient's intrathecal space at the appropriate spinal level, such as the thoracic spinal level for axial back pain. In a preferred embodiment, the electrical lead is introduced percutaneously although the electrical lead can also be introduced via open surgery. Preferably, the lead seals the hole made in the dura during passage of the lead intrathecally to prevent leakage of CSF. For axial back pain, the electrical lead is positioned at the level of a thoracic region of the spinal cord such as T-7 to T-12. In a preferred embodiment, the lead is positioned at levels T-7 or T-8 for axial back pain. The electrical lead is placed intrathecally and this includes placing the electrical lead in or underneath the dura mater. This includes placing the lead between the dura mater and the arachnoid mater as well as placing the lead under the arachnoid mater (subarachnoid space placement) between the arachnoid mater and the pia mater. Preferably, the electrical lead is closer to the pia mater than the subarachnoid mater and more preferably is in direct contact with the pia mater. This allows the electrical lead to be as close as feasibly possible to the spinal cord to achieve focal stimulation of the desired spinal region.

The electrical lead can be a cylindrical lead with circumferential stimulating electrodes on the distal portion thereof or a paddle-style electrical lead that has a distal paddle shaped body with stimulating electrodes disposed thereon. In certain embodiments, the lead, such as a percutaneous lead, has features that position the lead electrodes as close as possible to the pia mater. Preferably, the electrodes are substantially in contact with the pia mater. In certain embodiments, the lead, such as a percutaneous lead, has features that prevent rotational, lateral movement, or posterior/anterior movement. Such features apply an outward radial force to tissue in the intrathecal space. In certain embodiments, the electrodes are partially covered and partially exposed. The exposed portion of the electrodes is directed towards the spinal cord in an operative configuration. In certain embodiments, the lead has a positioning device as described in co-pending U.S. application entitled: “Electrical Lead Positioning Systems and Methods” (Ref. No. NAT-021916-US-ORD), filed on Mar. 15, 2013 and incorporated by reference in its entirety herein. In particular, the lead can have a positioning device located on the lead body comprising at least three arms radiating from the lead body, wherein the angle between at least two of the arms is greater than 120 degrees. In certain embodiments, a positioning device has arms that point in the distal direction to allow “pullback” into a touhy needle or other introducer without shearing the arms off. Arms of a positioning device can have superelastic reinforcing materials within them, such as nitinol wire or MP35N or platinum iridium coil. Such materials can add stiffness to the arms, thereby increasing the outward radial force the arms exert in an operative configuration. The arms of a positioning device push away from the dura and move the lead closer to the spinal cord but do not push off the spinal cord in an operative configuration. There can also be radiopaque markings differentiating the arms such that the physician may determine the rotational orientation under fluoroscopy, particularly for determining the electrodes' orientations if they are partially covered. Preferably, the lead body is substantially made from silicone.

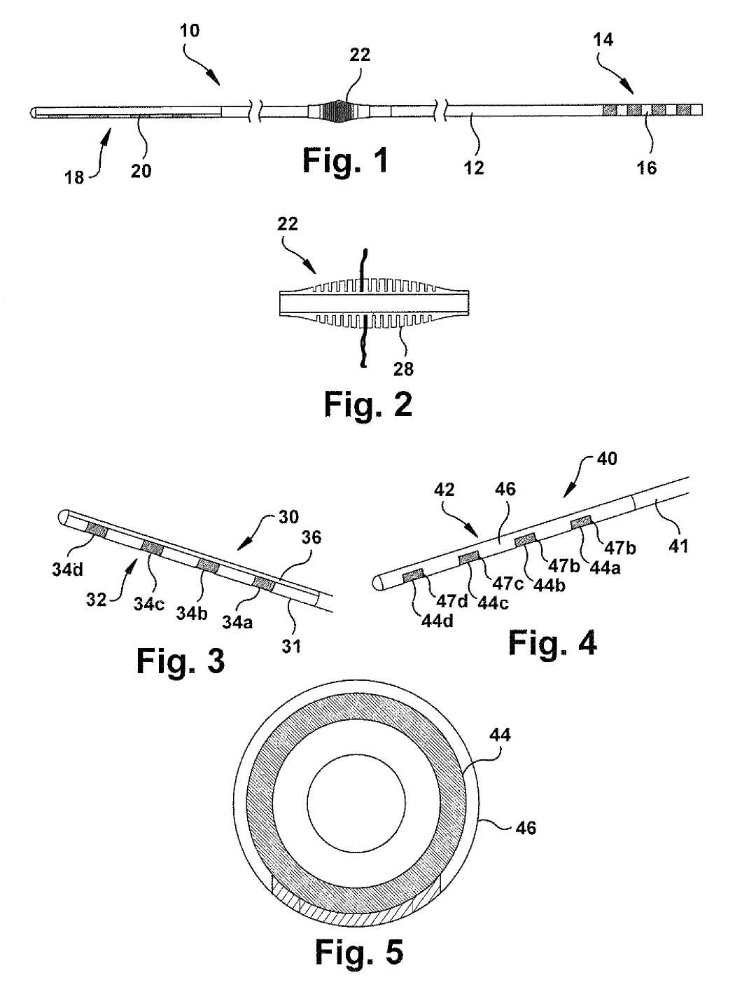

Referring to FIG. 1, in an embodiment, the present invention provides an electrical lead 10 comprising a lead body 12 having a proximal portion 14 comprising a plurality of electrodes 16 and a distal portion 18 comprising a plurality of electrodes 20. It should be noted that additional electrodes can be disposed on the lead body that are not part of the “plurality of electrodes” as that term is used herein. Electrical lead 10 further comprises an electrical conductor (not shown) extending between and electrically connecting the proximal plurality of electrodes 16 and the distal plurality of electrodes 20. Electrical lead 10 further includes a plug 22 located on lead body 12 between proximal portion 14 and distal portion 18 that defines a plurality of slots 28 as shown in FIG. 2. Preferably, plug 22 has a maximum outer diameter greater than the maximum outer diameter of lead body 12 and is sized to seal the aperture created in the dura upon insertion of the lead. In an operative configuration of electrical lead 10, dura mater as well as surrounding tissue can occupy portions of the slots 28 thereby plugging the aperture created in the dura from lead insertion.

An electrical lead can further include features to shield portions of the electrodes. For example, as illustrated in FIG. 3, an electrical lead 30 comprises a lead body 31 having a distal portion 32 comprising a plurality of electrodes 34 disposed thereon. The electrodes are disposed circumferentially around the lead body. Electrical lead 30 further includes a cover 36 coupled to distal portion 32 of lead body 31 and is disposed less than 360 degrees about lead body 31. In certain embodiments, cover 36 is disposed about 220 degrees about lead body 31. Cover 36 thus covers certain portions of electrodes 31 such that the remaining portions of electrodes 31 are exposed.

Referring to FIG. 4, in another embodiment, an electrical lead 40 comprise a lead body 41 having a distal portion 42 comprising a plurality of electrodes 44 disposed thereon. In a preferred embodiment, electrodes 44 having a smaller diameter than lead body 41. Electrical lead 40 further comprises a cover or overtube 46 that is coupled to distal portion 42 of lead body 41 and is disposed 360 degrees about lead body 31. In a preferred embodiment, the electrodes are recessed from the outer surface of the lead's distal portion. In particular, as seen in the cross-section of distal portion 42 in FIG. 5, electrodes 44 have a smaller diameter than the diameter of overtube 46 and are thus recessed in overtube 46. Overtube 46 defines apertures 47 that are aligned with electrodes 44 to expose certain portions of electrodes 44. Each recess may be between about 0.002 inches and about 0.020 inches. The recessing of the electrodes can help to reduce the spread of current to focus the stimulation on the desired region such as, but not limited to, low back fibers of the spinal cord for treatment of low back pain or focal areas of stimulation tailored to the individual patient's area of pain. In order to create the recessed electrodes, an electrode can be used with a smaller diameter than the diameter of the distal portion of the lead body. A reduced diameter distal portion can be manufactured using standard lead construction techniques such as molding or piece by piece constructions as depicted in FIG. 7.

in other embodiments as depicted in FIGS. 12 and 13, electrodes 70 having a raised surface 72 such that the electrodes are flush with or protrude from the overtube 74. When the electrodes protrude from overtube 74, each of the plurality of electrodes has a raised outer surface such that each of the electrodes has a maximum outer diameter greater than the maximum outer diameter of the overtube. The raised surface of each of the electrodes therefore protrudes out of the respective one of the plurality of apertures of the overtube.

In embodiments, where raised surface 72 protrudes from overtube 74, surface 72 effectively acts as a mechanical holding feature preventing rotation and separation of the electrode from the overtube. The raised surface 72 and the corresponding aperture in the overtube also help a clinician locate the electrode. This portion of the electrode may also be used to determine directionality of the lead under fluoroscopy. The raised surface can be seen when scanned perpendicularly using fluoroscopy. Additionally, the overtube may have a radiopaque marker located in the tip, or along the body to assist in locating the lead in the body. The marker in the overtube may be arranged so that it is approximately 90 degrees offset from the raised surface. This will allow a clinician to take a single fluoroscopic snapshot of the lead either in the anterior-posterior plane or lateral plane and be able to tell which direction the exposed portion of the electrodes is facing.

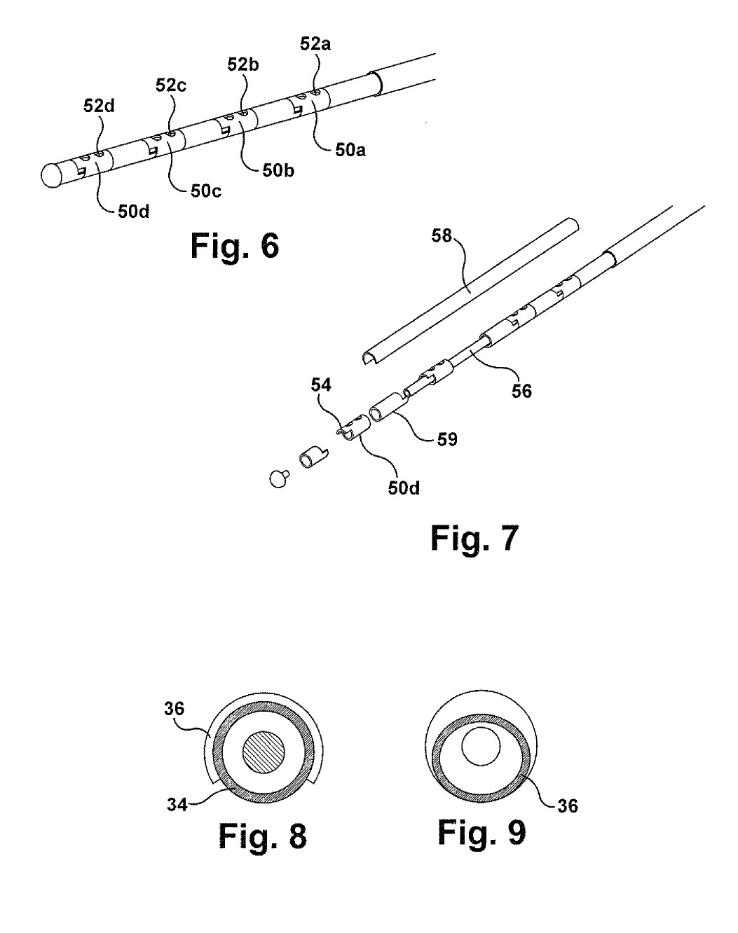

Referring to FIG. 6, in certain embodiments, electrodes 50 define holes or notches 52 in order to mechanically accept an adhesive that is used to couple a cover or overtube to the lead body of the lead. Such holes 52 increase the holding force of the distal portion of the lead. Each notch defines a bore extending in an axis different than the longitudinal axis of the lead body.

Each electrode can have a lip 54 with an inner surface to which an electrical conductor attaches. As such, the electrical conductor or conductors are sandwiched between the electrodes 50 and an inner tube 56 extending through the lumens of electrodes 50. Inner tube 56 also provides structural rigidity to the electrodes. FIG. 7 depicts a cover 58 that can be coupled to the distal portion of the lead body. FIG. 7 also depicts insulating separators 59 separating electrodes 50.

As seen in the cross-sectional views of FIGS. 8 and 9, the electrodes and the overtube or cover can have different configurations. For example, in FIG. 8, which is a cross-section of the electrical lead depicted in FIG. 3, electrode 34 is substantially cylindrical and is not recessed and has the same outer diameter of the lead body. FIG. 9 depicted an electrode 36 with a substantially oblong shape. In other embodiments, the electrodes have other shapes that are substantially non-cylindrical. Other cross sections include but are not limited to a rectangular cross-section or an eight-shaped cross-section. A non-cylindrical cross section may be preferential to help the lead from rotating after being implantation.

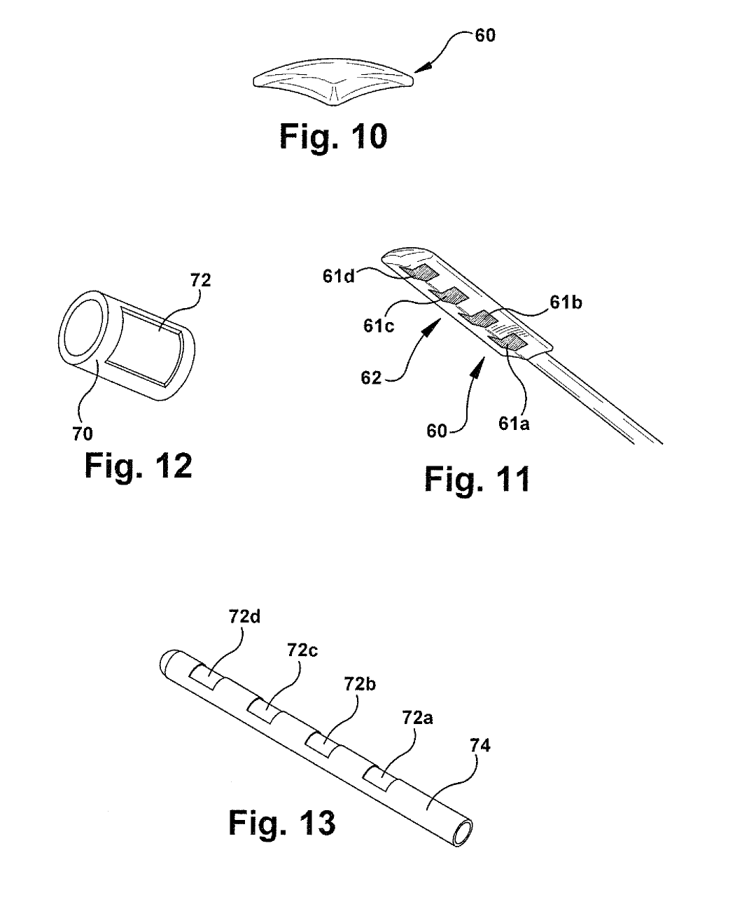

FIG. 10 is an end view of a paddle style lead 60 depicted in FIG. 11 that could be used for intrathecal stimulation. The stimulating surface 62 (the surface of the lead comprising electrodes 61) of paddle lead 60 has a curved configuration, such as a substantially V-shape, to match the configuration of the intradural space. Such a configuration minimized migration of the lead in the intrathecal space.

Preferably, the distal portion of the lead has a larger cross section in the x plane than the y plane for stabilization of the lead to prevent rotation after implantation.

In any of the embodiments described above and in other embodiments having only a portion of the electrodes exposed, the electrodes are shielded on one side of the lead body. Preferably, only about 45 to about 225 degrees of electrode surface area of an electrode is exposed. This allows the electrical current to be directed toward the spinal cord, reducing the amount of energy needed to obtain therapeutic amplitudes. The shielding of the electrodes also prevents spreading of the electric current to other portions of the spinal cord causing side effects. The focal field of the shielded electrode allows the user to more precisely stimulate a desired region of the spinal cord intrathecally while minimizing side effects and maximizing battery life for the device.

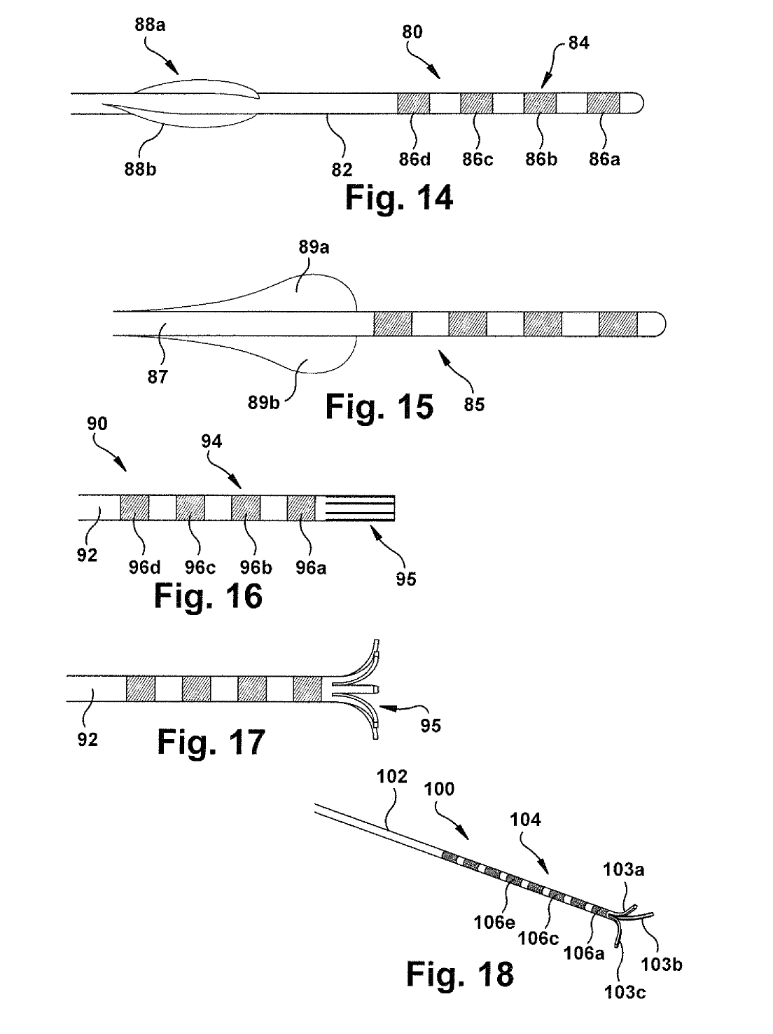

Referring to FIGS. 14 to 21, the present invention also provides electrical leads with features that stabilize the lead in the intrathecal space. For example, referring to FIG. 14, in certain embodiments, the present invention provides an electrical lead 80 comprising a lead body 82 having a distal portion 84 comprising a plurality of electrodes 86. Electrical lead 80 further comprises a fin 88 located on the lead body 82. Fin 88 can have any suitable configuration so long as the fin prevents or minimizes rolling of the lead off of the target site, such as the dura surrounding the spinal cord. In FIG. 14, electrical lead 80 has two fins 88, but could have more, on opposing sides of lead body 82 longitudinally spaced apart from one another. In FIG. 15, fins 89 are also on opposing sides of lead body 87 of lead 85 but have a greater width than fins 88 depicted in FIG. 14. Fins 89 collectively have a substantially pear-shape but could have other shapes as well.

Referring to FIGS. 16 and 17, in another embodiment, an electrical lead 90 comprises a lead body 92 having a distal portion 94 comprising a plurality of electrodes 96. Distal portion 94 also comprises a plurality of radially spaced arms 95 that radiate from lead body 92 or an intermediate structure coupled to lead body (such as a sleeve) disposed about the lead body. Arms 95 assume a compressed configuration as depicted in FIG. 16 when disposed within an outer sheath, and assume a relaxed or expanded configuration when the outer sheath is retracted as depicted in FIG. 17. In such a configuration and during placement of the lead in the intrathecal space, arms 95 apply radial forces against the dura to stabilize the lead in the intrathecal space. Arms 95 preferably angle forward so that the lead can be withdrawn back into a needle or other introducer during the surgical procedure without having a risk of shearing off on the sharp needle tip. In particular, in this embodiment, the plurality of arms is oriented radially outwards in a relaxed configuration. In certain embodiments, distal portion 94 comprises a distal end and the plurality of arms is disposed on the distal end of the distal portion as seen in FIG. 17. However, although arms 95 are depicted as being located on the distal portion of the lead body, they could be located on other regions of the lead body so long as they stabilize the lead in the intrathecal space.

Referring to FIG. 18, in certain embodiments, a lead 100 comprises a lead body 102 having a distal portion 104 comprising a plurality of electrodes 106. Lead 100 further comprises at least three arms 103 that radiate from lead body 102 or radiate from an intermediate structure disposed about lead body 102. At least two of the arms are separated by greater than 120 degrees. Such a positioning device is described in greater detail in co-pending U.S. application entitled: “Electrical Lead Positioning Systems and Methods” (Ref. No.: NAT-021916-US-ORD), filed on Mar. 15, 2013, and incorporated by reference herein. The at least three arms extending from the lead body having a compressed configuration when within an outer sheath and a relaxed configuration when the outer sheath is retracted. Distal portion 104 of lead body 102 has a distal end and the at least three arms 103 are disposed on the distal end of the distal portion in certain embodiments and as depicted in FIG. 18.

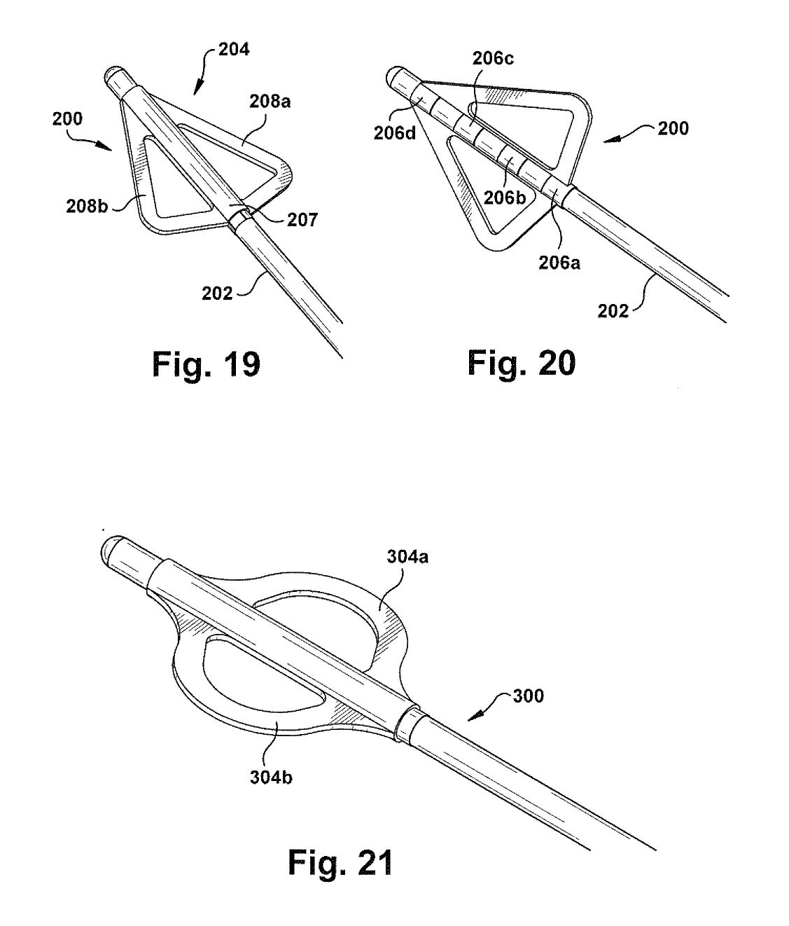

Referring to FIGS. 19 and 20, in another embodiment, a lead 200 comprises a lead body 202 having a distal portion 204 comprising a plurality of electrodes 206. Distal portion 204 also comprises a cover 207 coupled to distal portion 204. Cover 207 has at least one fin 208 and preferably two opposing fins that extend laterally from cover 206. As shown in FIG. 20, cover 207 is disposed on less than the entire outer surface of distal portion 204 such that a portion of electrodes 206 are exposed. FIG. 21 depicts another embodiment of a lead 300 having fins 304 that collectively have a substantially oval configuration. Other configurations are also possible. In these embodiments, the fin structures stabilize the lead in the intrathecal space to minimize lead rotation and mask part of the electrodes for focal stimulation as described above.

After the electrical lead has been positioned at the suitable level of the spinal cord, a method comprises delivering an electrical signal to the patient's spinal cord at the suitable level to treat the patient's medical condition or improve the patient's function. Because the lead is positioned intrathecally, the amplitude of the electrical signal can be lower than if the electrical lead were positioned epidurally. For example, in certain embodiment, the amplitude of the electrical signal is between about 0.1 to about 5 milliamps. In preferred embodiments, part of the lead's electrodes is shielded during delivery to direct the stimulation in a desired direction.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. A method of treating pain in a patient suffering therefrom comprising:
identifying a patient suffering from the pain;
inserting an electrical lead into the patient's body;
positioning the electrical lead in the patient's intrathecal space at a level of the spinal cord;
delivering an electrical signal to the patient's spinal cord at the spinal cord level; and
treating the pain in the patient, wherein the pain is axial back pain, groin pain or cancer pain.

2. The method of claim 1, wherein the pain is axial back pain, the electrical lead is positioned at the thoracic level of the spinal cord, and the electrical signal is delivered to the thoracic level of the spinal cord.

3. The method of claim 2, wherein the thoracic level is between levels T-7 and T-12.

4. The method of claim 3, wherein the thoracic level is level T-7 or T-8.

5. The method of claim 1, wherein the pain is groin pain.

6. The method of claim 1, wherein the pain is cancer pain.

7. The method of claim 1, wherein positioning an electrical lead in the patient's intrathecal space is placing the electrical lead in or underneath the dura mater.

8. The method of claim 1, wherein positioning an electrical lead in the patient's intrathecal space is placing the lead between the dura mater and the arachnoid mater.

9. The method of claim 1, wherein positioning an electrical lead in the patient's intrathecal space is placing the lead under the arachnoid mater between the arachnoid mater and the pia mater.

10. The method of claim 1, wherein positioning an electrical lead in the patient's intrathecal space is placing the electrical lead closer to the pia mater than the subarachnoid mater.

11. The method of claim 1, wherein positioning an electrical lead in the patient's intrathecal space is placing the electrical lead in direct contact with the pia mater.

12. The method of claim 1, wherein inserting the electrical lead into the patient is percutaneously inserting the electrical lead.

13. A method of improving sensory or motor function in a patient suffering from a medical condition comprising:
identifying a patient suffering from the medical condition;
inserting an electrical lead into the patient's body;
positioning the electrical lead in the patient's intrathecal space at a level of the spinal cord;
delivering an electrical signal to the patient's spinal cord at the spinal cord level to modulate a spinal cord pathway; and
improving the sensory or motor function in the patient, wherein the medical condition is neuropathic pain, nociceptive pain, stroke, traumatic brain injury, or ataxia.

14. The method of claim 13, wherein the spinal cord pathway is the lateral spinothalamic tract, the anterior spinothalamic tract, the fasciculus gracile, or the fasciculus cuneate and the medical condition is neuropathic pain.

15. The method of claim 13, wherein the spinal cord pathway is the lateral spinothalamic tract or the anterior spinothalamic tract and the medical condition is nociceptive pain.

16. The method of claim 13, wherein the spinal cord pathway is the dorsal spinocerebellar tract, the ventral spinocerebellar tract, the lateral corticospinal tract, the ventral corticospinal tract, the tectospinal tract, the rubrospinal tract, or the vestibulospinal tract and the medical condition is stroke or TBI.

17. The method of claim 13, wherein the spinal cord pathway is the dorsal spinocerebellar tract or the ventral spinocerebellar tract and the medical condition is ataxia.
